(19) United States
(12) Patent Application Publication
(10) Pub. No.: US 2022/0087508 A1
(43) Pub. Date: Mar. 24, 2022

(54) ENDOSCOPE

(71) Applicant: OLYMPUS WINTER & IBE GMBH, Hamburg (DE)

(72) Inventor: Alrun THUEMEN, Hamburg (DE)

(73) Assignee: OLYMPUS WINTER & IBE GMBH, Hamburg (DE)

(21) Appl. No.: 17/419,488

(22) PCT Filed: Dec. 4, 2019
(86) PCT No.: PCT/EP2019/083729

§ 371 Date: Jun. 29, 2021

(30) Foreign Application Priority Data
Jan. 4, 2019 (DE) 10 2019 100 136.6

(51) Int. Cl. A61B 1/00

(52) U.S. Cl. CPC A61B 1/00105; A61B 1/00128

(57) ABSTRACT

An endoscope including a first substantially hollow cylindrical body and a second substantially hollow cylindrical body, wherein the first body and the second body at least partially overlap coaxially, and wherein between the first body and the second body a round cord seal is disposed, which is sealingly disposed between a first sealing surface of the first body and a second sealing surface of the second body. Wherein, the first sealing surface is conical.

### CROSS-REFERENCE TO RELATED APPLICATION

The present application is based upon and claims the benefit of priority from PCT/EP2019/083729 filed on Dec. 4, 2019, which claims priority to DE 10 2019 100 136.6 filed on Jan. 4, 2019, the entire contents of each of which is incorporated herein by reference.

### BACKGROUND

### Field

The present disclosure relates to an endoscope and more particularly to an endoscope with a first substantially hollow cylindrical body and a second substantially hollow cylindrical body, wherein the first body and the second body at least partially overlap coaxially, and wherein a round cord seal is disposed between the first body and the second body, which is sealingly received between a first sealing surface of the first body and a second sealing surface of the second body.

### Prior Art

Endoscopes have long been used in medicine to examine and/or treat cavities in the body of a human or animal patient that are difficult to access. For this purpose, endoscopes generally have an elongated shaft which is inserted through a natural or surgically created body orifice into the cavity to be examined. A main body is located at a proximal end of the shaft, which is used to handle the endoscope.

At a distal end of the shaft, an objective lens is usually provided, which produces an image of the cavity to be examined. This image is either transmitted via optical image conductors into the main body, where it is made available for observation by means of an ocular, or it is converted by means of electronic image converters into electrical video signals, which are transmitted by means of electrical lines or wirelessly into the main body, from where they are made available externally, possibly after electronic preprocessing. This can again be done via electrical lines or wirelessly.

Known endoscopes are generally constructed from several essentially hollow cylindrical bodies. In this context, the term “essentially hollow cylindrical” is not to be understood strictly geometrically, but is also intended to include such bodies that have a non-circular and/or changing cross-sectional profile in the direction of a longitudinal axis.

To protect the optical and/or electronic components of the endoscope from penetrating moisture or contamination, the interior of the endoscope is sealed. For this purpose, adjacent bodies of the endoscope casing are configured to partially overlap coaxially, wherein a round cord seal is provided between the bodies.

Round cord seals, also known as O-rings, consist of a closed cord of an elastic material with a round cross-section. They are usually first inserted into a circumferential groove in the outer periphery of the inner body before the outer body is slid over the inner body. In this process, the round cord seal is compressed between the groove base of the inner body and the inner periphery of the outer body, so that it lies sealingly against both surfaces.

While corresponding round cord seals have proven themselves in the prior art, they do have certain shortcomings.

For example, the round cord seal is sheared during assembly of the outer body. The greater the radial compression of the round cord seal is, the greater is the shear force. To prevent destruction of the round cord seal during assembly, the radial compression must therefore be kept small, which, however, limits the sealing effect of the round cord seal.

Likewise, the described structure of the seal is very large in a radial direction. Therefore, often only a round cord seal with a small cord thickness can be used, which also limits the sealing effect.

### SUMMARY

It is therefore an object to provide an endoscope which is improved with respect to the problems described above.

Such object can be achieved by an endoscope with a first substantially hollow cylindrical body and a second substantially hollow cylindrical body, wherein the first body and the second body at least partially overlap coaxially, and wherein a round cord seal is disposed between the first body and the second body, which is sealingly received between a first sealing surface of the first body and a second sealing surface of the second body, which is further configured in that the first sealing surface is conical.

Due to the conical configuration of the first sealing surface, the round cord seal is better supported during assembly, and the shear stress on the round cord seal is reduced. This enables higher compression of the round cord seal without having to fear destruction of the round cord seal due to shear forces. The higher compression simultaneously increases the sealing effect of the round cord seal.

The second sealing surface may be configured as a sealing shoulder with a substantially radial first partial surface and a substantially axial second partial surface. In this case, the supporting forces of the two partial surfaces may add up such that a resulting supporting force is exactly perpendicular to the conical first sealing surface. In this case, the round cord seal is practically no longer subjected to shear forces, and can thus withstand a particularly high compression.

The first sealing surface may have a taper ratio between about 1.4:1 and 2.4:1. At a taper ratio of 1.4:1, an angle between the first sealing surface and a longitudinal axis of the first body corresponds to about 35°. At a taper ratio of 2.4:1, the angle between the first sealing surface and the longitudinal axis of the first body corresponds to about 50°. With a taper ratio of the first sealing surface of 2:1, the angle between the first sealing surface and the longitudinal axis of the first body is 45°.

The first and the second sealing surfaces may be axially braced against each other, i.e. in the direction of the longitudinal axis of the first and second bodies. The compression of the round cord seal is then determined by the axial tightening force and may be dimensioned according to the requirements for the sealing effect.

Herein, the first body or the second body may comprise a thread, the endoscope may further comprise a nut cooperating with the thread to brace the first sealing surface and the second sealing surface axially against each other. In this way, the axial bracing may be adjusted particularly easily. The thread may be a fine thread.

The radial first partial surface may be formed by a separate sealing ring. This provides further degrees of freedom for mounting the seal.

A gap seal may be disposed preceding the round cord seal, where “preceding” can be understood as meaning that, in the metering direction from the outer area exposed to humidity and/or contamination to the inner area of the endoscope to be kept dry and clean, the gap seal is arranged first and then the round cord seal. If suitably dimensioned, a gap seal may prevent liquids such as water and/or aggressive processing chemicals from advancing as far as the round cord seal and damaging it.

In the first body and/or the second body of the endoscope, optical components may be arranged. These may be part of an objective lens, a relay lens system, and/or an ocular.

Likewise, in the first body and/or the second body of the endoscope, electrical and/or electronic components may be arranged. These may be, for example, electronic image converters, electromagnetic actuators, signal lines, matching circuits, and/or antennas for wireless data transmission.

### BRIEF DESCRIPTION OF THE DRAWINGS

The embodiments are described in more detail below with reference to a number of exemplary drawings, wherein the illustrated embodiments are merely intended to provide a better understanding without limiting the scope of protection of the appended claims, in which:

FIG. 1 illustrates an endoscope,

FIG. 2 illustrates a simplified sectional view of a round cord seal of an endoscope,

FIG. 3 illustrates a simplified sectional view of another round cord seal, and

FIG. 4 illustrates a simplified sectional view of still another round cord seal.

### DETAILED DESCRIPTION

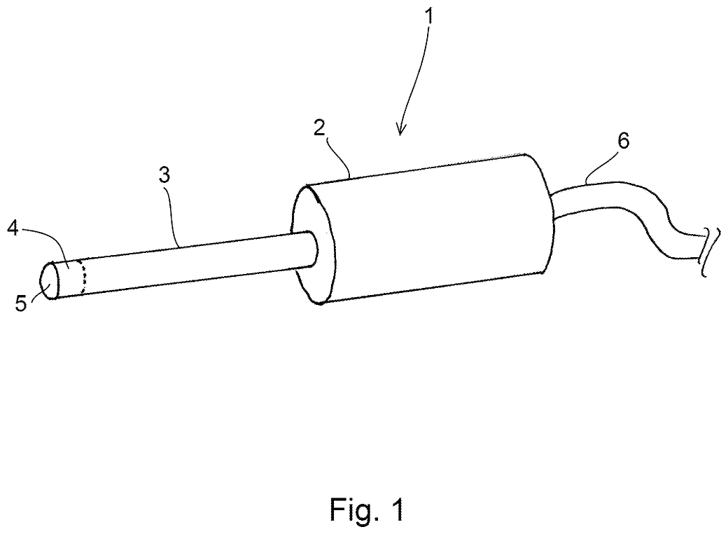

FIG. 1 shows an endoscope 1. The endoscope 1 comprises a main body 2 and an elongated shaft 3. An objective lens 4 is disposed at the distal end of the shaft 3, and the shaft 3 is closed at the distal end by a hermetic window 5.

The endoscope 1 is a video endoscope. The image of the objective lens 4 is converted by an electronic image converter, which is not shown, into electrical video signals which, after internal preprocessing, are made available via a cable 6 for display and/or further evaluation.

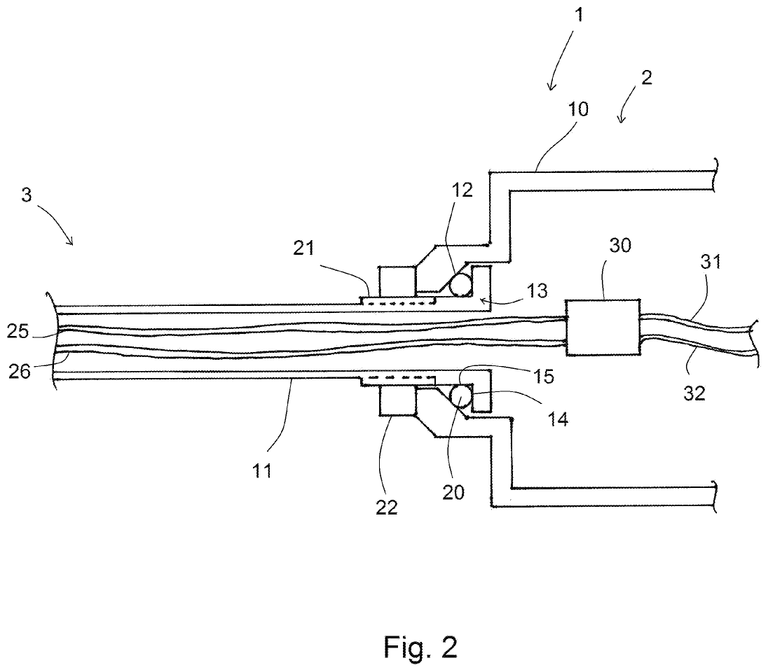

FIG. 2 shows a seal between two hollow cylindrical bodies 10, 11 of the endoscope 1. Here, the body 10 is part of the main body 2 of the endoscope 1, and the body 11 is part of the shaft 3 of the endoscope 1.

The body 10 comprises a conical sealing surface 12, which has a taper ratio of approximately 2:1. The body 11 comprises a sealing surface in the form of a sealing shoulder 13, which consists of a radially arranged first partial surface 14 and an axially arranged second partial surface 15.

A round cord seal 20 is disposed between the sealing surface 12 and the sealing shoulder 13. In order to press the round cord seal firmly against the sealing surface 12 and the sealing shoulder 13, the body 10 and the body 11 are braced axially against one another. For this purpose, the body 11 comprises an external thread 21 onto which a nut 22 is screwed. Tightening the nut 22 tightens the body 11 in the direction of its longitudinal axis against the round cord seal 20. The conical sealing surface 12 in turn presses the round cord seal 20 in the direction of the partial surface 15 of the sealing shoulder 13. The round cord seal 20 is thereby pressed firmly against the respective sealing surfaces without any significant shear forces acting.

In addition to its clamping function, the thread 21 and the nut 22 act as a gap or labyrinth seal which prevents liquid from advancing to the round cord seal. In particular, the round cord seal can thus be protected from the effects of aggressive cleaning chemicals.

Within the second body 11, electrical lines 25, 26 are installed which conduct the electrical video signal of an electronic image converter, which is not shown, to a preprocessing circuit 30. From the preprocessing circuit 30, the processed video signal is routed out of the endoscope 1 via further lines 31, 32. The lines 31, 32 are placed in the cable 6.

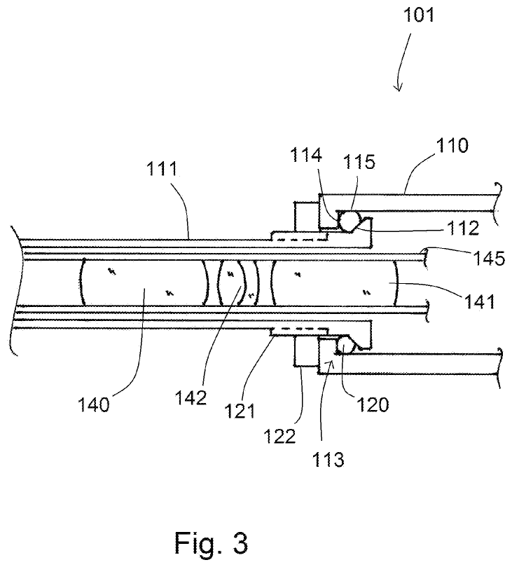

FIG. 3 shows an alternative sealing between two hollow cylindrical bodies 110, 111 of an endoscope 101. Here, the body 110 is part of a main body of the endoscope 101, while the body 111 is part of a shaft of the endoscope 101.

In the present embodiment, the body 111 comprises a conical sealing surface 112, while the body 110 comprises a sealing shoulder 113 having a radial partial surface 114 and an axial partial surface 115.

A round cord seal 120 is again disposed between the sealing surface 112 and the sealing shoulder 113. For axial clamping of the bodies 110, 111, a thread 121 is provided on the body 111, as in the embodiment example of FIG. 1, onto which a nut 122 is screwed. Thread 121 and nut 122 in turn additionally act as a gap or labyrinth seal.

The endoscope 101 is configured as an optical endoscope. Therefore, a relay lens system is disposed inside the endoscope 101, which transmits an intermediate image generated by a lens to an ocular. Two rod lenses 140, 141 and an achromat 142 disposed therebetween are shown as part of the relay lens system. The relay lens system is supported by a system tube 145.

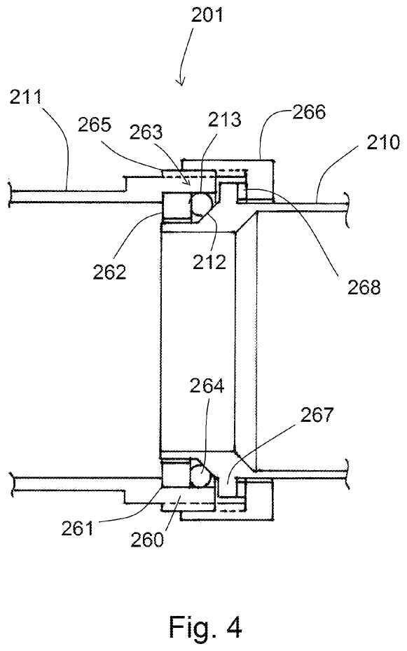

FIG. 4 shows another possible embodiment of a round cord seal of an endoscope 201, wherein a body 210 is sealed against a body 211. Here, the body 211 has a wall thickness too small to form a sealing shoulder.

The structure of the seal is therefore as follows: a conical sealing surface 212 is disposed on the body 210, and the body 211 comprises a collar 260 which forms an axial sealing surface 213. Adjacent to the collar 260 in the axial direction a stop shoulder 261 is disposed, which however is too short to function as a sealing shoulder.

Therefore, an additional sealing ring 262 is inserted into the collar 260, which abuts against the stop shoulder 261 and forms a sealing shoulder 263 together with the collar 260. A round cord seal 264 is again disposed between the sealing surface 212 and the sealing shoulder 263.

For bracing the bodies 210 and 211, a thread 265 is disposed on the collar 260, onto which a union nut 266 is screwed. The union nut engages around a flange 267 disposed on the body 210.

A sliding ring 268 may be provided between the union nut 266 and the flange 267.

In the above embodiments, the endoscopes 1, 101, 201 are shown in a highly simplified manner to facilitate understanding of such embodiments. In fact, modern endoscopes are much more complicated in design. Also, the configuration of round cord is only one of several sealing concepts used in modern endoscopes. Particularly sensitive components of such endoscopes may be disposed in hermetically sealed sections of the endoscope which are, for example, welded, soldered or cast.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. An endoscope comprising:
a substantially hollow cylindrical first body; and
a substantially hollow cylindrical second body,
wherein the first body and the second body at least partially overlap coaxially,
a round cord seal is disposed between the first body and the second body,
the round cord seal is sealingly received between a first sealing surface of the first body and a second sealing surface of the second body, and
the first sealing surface is conical.

2. The endoscope according to claim 1, wherein the second sealing surface is configured as a sealing shoulder with a substantially radial first partial surface and a substantially axial second partial surface.

3. The endoscope according to claim 1, wherein the first sealing surface has a taper ratio between 1.4:1 and 2.4:1.

4. The endoscope according to claim 3, wherein the first sealing surface has a taper ratio of 2:1.

5. The endoscope according to claim 1, wherein the first sealing surface and the second sealing surface are axially braced against each other.

6. The endoscope according to claim 5, wherein one of the first body or the second body comprises a threaded surface, and that the endoscope further comprises a nut mating with the threaded surface to brace the first sealing surface and the second sealing surface axially against each other.

7. The endoscope according to claim 2, wherein the radial first partial surface is formed by a separate sealing ring.

8. The endoscope according to claim 1, further comprising a gap seal disposed to proceed the round cord seal.

9. The endoscope according to claim 1, further comprising optical components disposed in one or more of the first body and the second body.

10. The endoscope according to claim 1, further comprising electrical and/or electronic components disposed in one or more of the first body and the second body.
